(19) United States
(12) Patent Application Publication
(10) Pub. No.: US 2003/0022382 A1
(43) Pub. Date: Jan. 30, 2003

(54) Apparatus for the analysis for blood samples

(76) Inventor: Kent M. Negersmith, Kent Lakes, NY (US)

Correspondence Address: LAWRENCE CRUZ, 12 GOOD HILL RD., BETHEL, CT, 06801, US

(21) Appl. No.: 10/202,432

(22) Filed: Jul. 25, 2002

Related U.S. Application Data
(60) Provisional application No. 60/308,408, filed on Jul. 26, 2001.

(51) Int. Cl. G01N 33/86; B01L 3/02

(52) U.S. Cl. 436/70; 436/174; 436/180; 422/73; 422/99; 422/102; 422/940; 422/941; 422/100

(57) ABSTRACT

A system for handling and testing fluids and gases such as, for example, blood samples, includes a tube in cooperation with at least one input port and at least one pressure source mounted in a novel arrangement to enable precise, accurate, and relatively spill-proof handling of fluids and gases to perform mixing and visual inspection and other tests. After testing, various components may be easily disassembled and discarded. A method of operation includes energizing the pressure source, in the form of a flexible bulb, to draw blood from a standard blood tube into a measuring tube according to a preferred embodiment of the invention said system in order to visually inspect sediment or red blood cell settling level after a predetermined time in order to test for certain blood characteristics.

### RELATED APPLICATION

This application relates to and claims priority from U.S. PROVISIONAL APPLICATION No. 60/308,408 filed on Jul. 26, 2001.

### TECHNICAL FIELD OF THE INVENTION

The present invention relates to fluid handling systems and related methods and, more particularly, to systems and methods for sampling, mixing, measuring or testing fluids, especially in a laboratory setting. One specific use for which the invention is particularly well-suited is the measurement of the amount of relatively dense particles within a liquid sample.

### BACKGROUND OF THE INVENTION

While the present invention has broad utility and is suitable for a variety or uses with a variety of different substances, the preferred embodiments described herein are particularly well-suited for use in handling and testing blood samples. By way of example, and not intended to be limiting, the present invention is described herein with respect to blood sampling, handling and testing.

Various blood tests require removing the stopper or cap from a blood containing tube and then removing a portion of sample, diluting and mixing with a reagent, and placing into a readout instrument. These tests are either special tests which are not performed automatically or tests that require manual manipulation of the plasma, serum, or whole blood. Typically these tests are for chemistry, immunology, and hematology analysis.

The present invention is adaptable to a number of blood tests. One test which is particularly adaptable to is the “Erythrocyte Sedimentation Rate” (ESR) test. There are several methods for performing this test but the most common and preferred primary method is the “Westergren Method”

A blood sample which has been diluted by 20% with anti coagulant is put into a 200 mm long by 2.5 mm inside diameter rigid tube. This tube is placed vertically on a stand, then the interface of plasma and red cells is measured after one hour in millimeters of settling of red cells. Normal samples range between 0 and 15 mm of settling. Abnormals can settle over 100 mm in one hour.

The rate of erythrocyte sedimentation has long been known to be increased by the presence of acute phase proteins, especially fibrinogen, alpha-2 macroglobulin and, to a lesser extent, immunoglobulins. Some serum proteins, notably albumin, have also been reported to decrease erythrocyte sedimentation. These serum proteins appear to act by reducing (or, for albumin, increasing) the electrostatic forces between red cells, allowing rouleaux to form. This aggregation allows the red cells to sediment through the plasma more quickly. The ESR is therefore increased by conditions that increase the concentration of acute phase proteins in the plasma, such as acute inflammation, fever and infections. The use of the ESR in diagnosis and monitoring of temporal arteritis and polymyalgia rheumatica is particularly well defined. Symptoms of many acute phase reactions can be non-specific (general malaise or musculoskeletal complaints), and performance of the ESR can aid in distinguishing inflammatory causes from other possible causes of these symptoms. The ESR is less useful as a general screening for wellness in the absence of symptoms, although this use is not uncommon. Long term conditions, such as autoimmune diseases (rheumatoid arthritis) or cancer can also increase the ESR, and so the ESR is often used to help diagnose and monitor disease activity and response to treatment in these conditions.

When performing the ESR test, typically the laboratory technician does the following:

1. Takes the sample tube off a rocker mixer and removes the sample tube cap.

2. Using a disposable pipette aspirates out enough sample (approx. 1.5 ml) and dispenses it into a plastic vial until it reaches a fill mark near the top of the vial. Note: there are two types of vials. One is empty and the other has sodium citrate reagent.

3. The vial is then capped and mixed.

4. The Westergren tube (W.T.) is pushed into the vial (the vial's inside diameter is the same as the outside diameter of the W. T). The sample rises up the W. T. and is adjusted to the zero mark near the top of the tube by adjusting the tube up or down.

5. Places the tube on a holding rack.

6. Recaps the blood sample.

7. Reads ESR by eye one hour later.

8. Disposes of the vial, W.T., and pipette.

During this procedure blood samples commonly contaminate safety gloves from the top of the open sample tube or from the sample cap. The samples also commonly contaminate the outside of the sample tubes. The contaminant is spread around the laboratory via gloves to benches, computers, etc. The disposables often leak into the waste containers. At times samples leak out of the vial while filing the W. T. As a result, all samples must be treated as if the patient's sample is contaminated.

### OBJECTS OF THE PRESENT INVENTION

One object of the present invention is to eliminate the potential for contamination hazards.

Another object of this invention is to provide a new means for sampling directly from the sample tube without the need to remove the stopper or cap.

Another object of this invention is to provide means to proceed or follow the sample through the same conduit with reagent and/or air for dilution and mixing.

### SUMMARY OF THE INVENITON

A system for handling fluids and gases such as, for example, blood samples, comprises a tube in cooperation with at least one input port and at least one pressure source mounted in a novel arrangement to enable precise, accurate, and relatively spill-proof handling of fluids and gases to perform mixing and visual inspection and other tests. After testing, various components may be easily disassembled and discarded. A method of operation includes energizing the pressure source, in the form of a flexible bulb, to draw blood from a standard blood tube into a measuring tube according to a preferred embodiment of the invention said system in order to visually inspect sediment or red blood cell settling level after a predetermined time in order to test for certain blood characteristics.

### BRIEF DESCRIPTION OF THE DRAWINGS

FIG. 1 is a schematic side view of a pipette with port and pressure bulb assembled according to a first embodiment of the present invention.

FIG. 2 is a detailed, partial, schematic side view of a first end of the pipette shown in FIG. 1.

FIG. 3 is a schematic side view of a pipette with port and pressure bulb assembled and secured within a holder rack according to a first embodiment of the present invention.

FIG. 4 is a detailed, partial, schematic side view of a pipette with port and pressure bulb assembled and secured within a holder rack and having a sample tube holder secured thereto according to a first embodiment of the present invention.

FIG. 5 is a detailed, partial, schematic front view of the sample tube holder, with a cut off section of the sample inlet tube, according to a first embodiment of the present invention.

FIG. 6 is a detailed, partial, schematic top view of the sample tube holder of FIG. 5.

FIG. 7 is a schematic side view of a pipette with port and pressure bulb assembled and secured within a holder rack and having a sample tube connected in fluid cooperation therewith according to a first embodiment of the present invention.

FIG. 8 is a schematic as in FIG. 7, having the sample tube removed and the fluid contents of the sample tube of FIG. 7 being now transferred into the pipette of FIG. 8.

FIG. 9 is a schematic side view of a pipette with two ports and pressure bulb assembled according to a second embodiment of the present invention.

FIG. 10 is a detailed, partial, schematic side view of a first end of the pipette shown in FIG. 9.

FIG. 11 is a schematic side view of a pipette with port and pressure bulb assembled and secured within a holder rack according to a second embodiment of the present invention.

FIG. 12 is a schematic front view of the system as shown in FIG. 11.

FIG. 13 is a schematic side view of a pipette with port and pressure bulb assembled and secured within a holder rack according to a second embodiment of the present invention.

FIG. 14 is a schematic side view of a pipette with port and pressure bulb assembled and secured within a holder rack and having a sample tube connected in fluid cooperation therewith according to a second embodiment of the present invention.

FIG. 15 is a schematic of the system shown in FIG. 14, further showing sample fluid partially drawn into a mixing chamber according to a second embodiment of the present invention.

FIGS. 16-20 are schematic views of the system shown in FIG. 15 having the sample tube removed and fluid/air in various states of mixing in a mixing chamber.

FIG. 21 is a schematic of the system shown in FIG. 14, further showing sample fluid drawn into a pipette according to a second embodiment of the present invention.

FIG. 22 is a schematic side view of a pipette with two ports, two pressure bulbs and a mixing chamber assembled according to a third embodiment of the present invention.

FIG. 23 is a schematic side view of a holder assembly according to a third embodiment of the present invention.

FIG. 24 is a schematic side view of the pipette assembly of FIG. 22 positioned in the holder assembly according to FIG. 23.

FIG. 25 is a schematic side view according to FIG. 24 and having a sample tube held therein in fluid communication.

FIGS. 26-28 are schematic views of the system shown in FIG. 25 having the sample tube removed and fluid/air in various states of mixing in a mixing chamber.

FIG. 29 is a schematic as in FIG. 22 having fluid contained in a mixing chamber.

FIG. 30 is a schematic side view of a pipette with three ports and a mixing chamber assembled according to a fourth embodiment of the present invention.

FIG. 31 is a schematic side view of a holder assembly according to FIG. 30 held in a holder assembly according to a fourth embodiment of the present invention.

### DESCRIPTION OF THE PREFERRED EMBODIMENTS

In a first embodiment, the invention is described relative to performing a modified Westergren Erythrocyte Sedimentation Rate Test.

### Method A

Samples are collected with sodium citrate reagent within the sample tube prior to collecting sample.

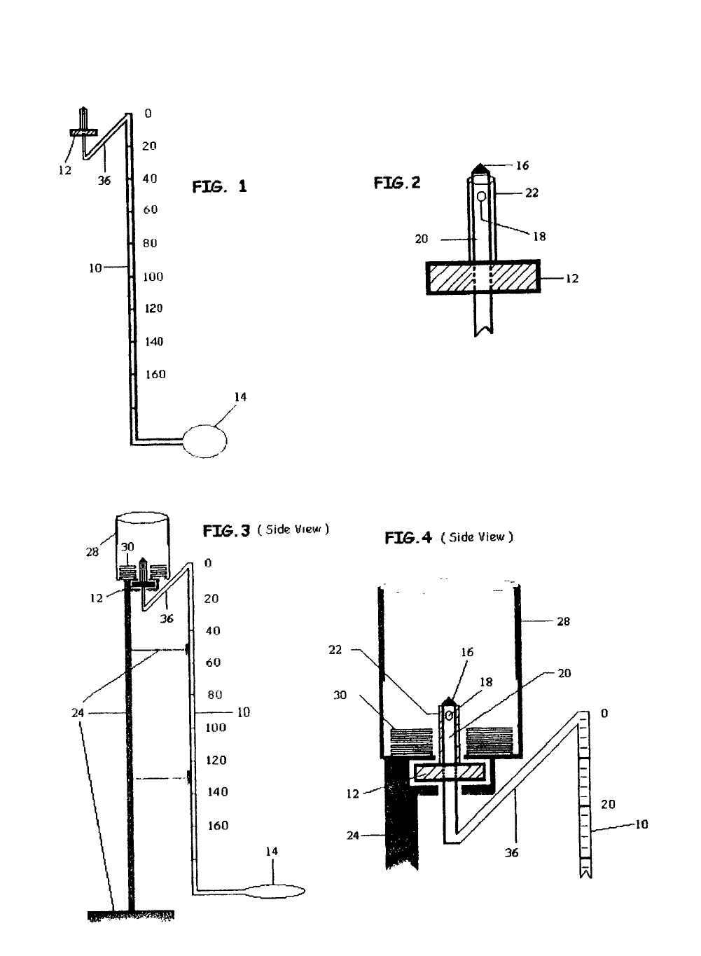

FIG. 1 shows a side view of the 200 mm long by 1.0 mm inside diameter rigid tube 10 with a sampling tab 12 on the upper inlet end and a flexible pipette bulb 14 on the lower end.

FIG. 2 shows the sampling end in detail. The stopper piercing tip 16 is sealed. The sample inlet port 18 is shown on the upper side of the inlet tube 20. A flexible silicone tube/sleeve 22 surrounds the sample inlet port 18. This sleeve 22 covers the entire inlet tube and terminates on the top of the sampling tab 12.

FIG. 3 shows a side view of the modified ESR tube 10 connected to a holding rack 24. The holding rack 24 has a slot 26 to hold the sampling tab 12 in place. This rack 24 also has a sample tube holder 28. The drawing shows that a technician has placed the ESR tube 10 on the holding rack 24 and has “charged” the aspiration pipette 14 by squeezing the pipette bulb 14. As the bulb 14 is squeezed the air pressure opens the silicone sleeve 22 and the air is released through the sample inlet port 18. The flexible silicone tube 22 returns and again seals the sample inlet port 18.

FIG. 4 shows a detailed side view of the sample tube holder 28 which is permanently mounted to the ESR tube 10 Rack/Base stand 24. This Rack/Base stand 24 will have multiple positions, each position will also have a sample tube holder 28. The drawing also shows a partial upper section of the ESR tube 10. Of particular importance are the springs 30 mounted in the bottom of the sample tube holder. Several of these springs 30 surround the sample inlet tube 20.

The purpose of these springs 30 is to assist in the removal of the sample tube 32. With no assistance, the sample inlet tube 20 will resist the sample tube stopper 34 removal, which poses the risk of suddenly popping off the stopper 34 in an uncontrolled manner as the technician removes the sample tube 32.

The technician will now insert the mixed sample tube 32 (See FIG. 7) pushing slightly until the stopper 34 bottoms out against the compressed springs 30. Also while inserting the sample tube 32 the flexible silicone tube 22 will be pushed down below the sample tube stopper 34. The sample inlet port 18 is now open to the sample which is pulled by vacuum in the flexible pipette 14 through the ESR tube 10 until it reaches the bottom of the ESR tube 10. Then the technician removes the sample tube 32 (See FIG. 8). The flexible silicone sleeve 22 will spring back up sealing off the sample inlet port 18, preventing further flow. This filling process will take several seconds. The ESR result is read one hour later.

There are various methods using various lengths and inside diameters, some on specific angles. Some of these methods are measured earlier, twenty minutes or so. Although the Westergren method is described herein, this system is adaptable to other methods.

FIG. 8 also shows a manual tubing pincher 11 mounted on the base stand 24 positioned below the 200 millimeter position of the ESR tube 10. This may be desirable to prevent red blood cells from settling beyond the ESR tube 10 bottom, towards the sampling pipette 14. Also shown is a flexible section of conduit 15 prior to the sampling pipette 14. Following the filling of the ESR tube 10 the Technician would push the flexible section conduit 15 down into the tubing pincher 11. If the overall instrument is further automated, a programmed pinch valve would be used.

Shown in FIG. 4 is the riser tube 36 which has several purposes. First, it allows room for the ESR tube 10 to be in front for easy readability. 2. Also, because it is a one-piece, molded rigid tube, the 0 mm mark 38 can be easily located precisely at the sample inlet port 18 height. This will prevent plasma and/or red cells from settling in the 200 mm measurement path. 3. This riser tube 36 section can also be calibrated in millimeters and be at some ideal angle for the technician to read an earlier ESR to screen stat samples. It is well known that the red cells will settle faster when the ESR tube 10 is on an angle. Essentially, the plasma can flow more easily upwards as the red cells settle off the upper inside wall of the riser tube 36.

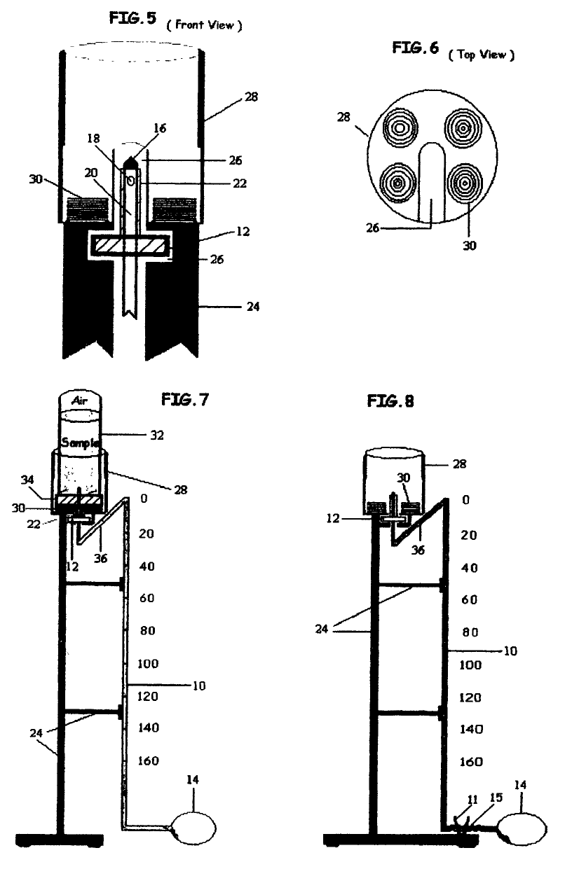

FIG. 5 shows an expanded front view of the sample tube holder 28, with a cut off section of the sample inlet tube 20. Part of the holding rack 24 is slotted to hold the sampling tab 12 in place and part of the upper sample tube holder 28 is slotted for the sample inlet tube 20 to fit into position. The depth of the sampling tab slot 26 is such that the inlet tube 20 will be centered in the sample tube holder 28.

Looking at FIG. 7 note that the flexible pipette bulb 14 is “nearly” back to its original round configuration. The volume of this pipette 14 is selected so as to control the flow of sample so that the ESR tube 10 is filled in several seconds.

Following the final readout the technician will remove the ESR tube 10 and discard it. Sample is contained via the silicone tubing sleeve 22 and the otherwise closed ESR tube 10.

### Method B

Many laboratories prefer to perform the ESR test using whole blood collected in tubes without sodium citrate diluent. Therefore, the technician must dilute the sample by 20% using sodium citrate reagent. Commonly they use an available method having a vial with liquid sodium citrate. The technician adds the whole blood sample to a line near the top of the vial. The sample is mixed and then the Westergren tube is pushed into it. At this point the technician follows the same steps as described above using a blood sample collected with the sodium citrate in the sample tube.

The present invention enables handling of these whole blood samples with dilution of the sample by 20% with sodium citrate.

FIG. 9 shows the same ESR tube 10 but the sampling tab 12 has two inputs. The vertical input 20 is identical to Method A, the horizontal input 40 is for the reagent addition and air addition. The air is for assuring accurate dilution and to mix the sample and reagent. The reagent inlet tube 40 also has a silicone (or other elastic) tube 42 that seals off the tube before and after use.

Between the two chambers there is a mark 46 inscribed, this mark is used to insure the proper volume of sample is aspirated. After the technician “charges” the flexible pipette 14 at the lower end of the ESR tube 10, at anytime thereafter he can aspirate a blood sample. As soon as the sample is inserted into the sample inlet tube 20 blood will begin flowing. The technician will remove the sample tube 32 when the sample reaches the mark 46. The time is several seconds for this filling to complete.

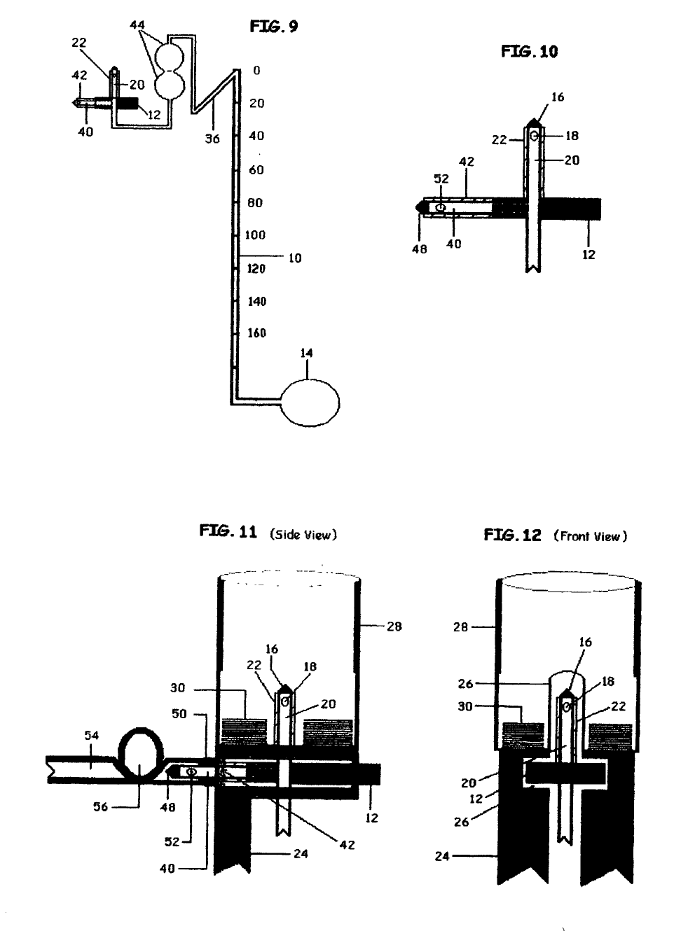

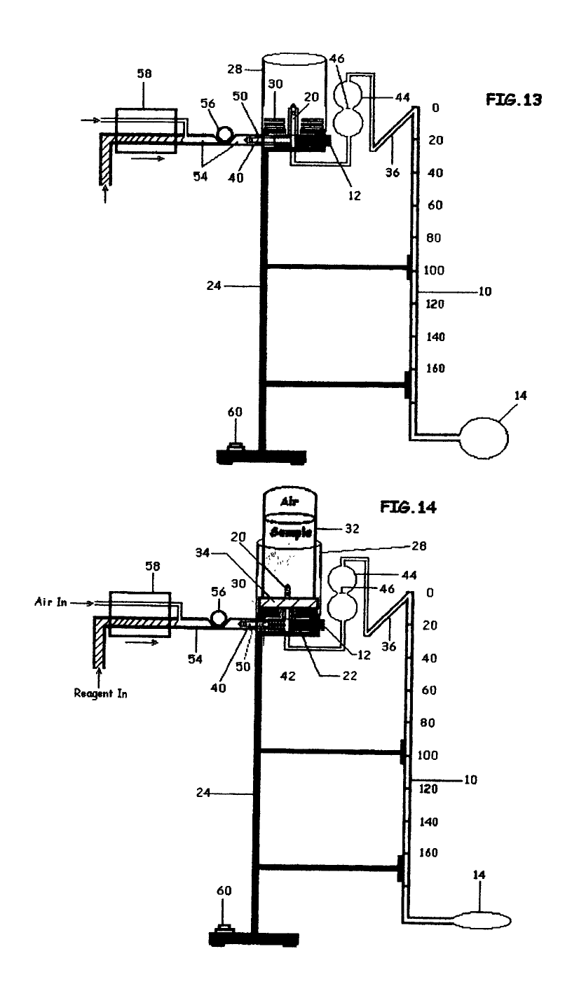

FIG. 10 shows the detail of the sampling tab 12. This tab 12 will fit into the sample tube holder 28 as shown in FIGS. 11 and 12. The sampling inlet tube 20 is identical to ESR method A. The reagent/air inlet tube 40 and tip 48 will slide through a hole 66 in the rear of the Rack/Base stand 24. There is a hard rubber seal 50 that the tip 48 and inlet tube 40 fit through. As the reagent/air inlet tube 40 is pushed into the base stand holder 24 the silicone tubing sleeve 42 will slide back and stay against the outside or front side of the base stand 24. The sample tube 20, silicone tubing sleeve 22 and reagent/air tubing sleeve 42 will rebound covering the inlet port 18 when the sample tube 32 is removed and port 52 when the sampling tab 12 is removed. The reagent/air inlet tube 40 is now open to a flexible tube 54 with an inside diameter larger than the outside diameter of the reagent/air inlet tube 40. This flexible tube 54 seals tightly around the hard rubber seal 50.

FIG. 11 shows a pinch valve 56 that seals off the path near the tip 48 of the reagent/air inlet tube 40. The leading end of this flexible tubing 54 where the reagent/air inlet port 52 is contained, is only air when a new ESR tube is installed. This pinch valve 56 prevents flow entering the sample inlet tube 20 during sample aspiration.

FIG. 12 is a front view and shows a notch 26 on the base stand 24 for the sampling tab 12 to slide into and a notch 26 in the front of the sample tube holder 28 for the sample inlet tube 20 to fit. When the sampling tab is fully inserted the sample inlet tube 20 will be centered inside the sample tube holder 28.

FIG. 13 shows the ESR tube 10 mounted to the “multiple” (only one position is shown) ESR sample tube holder 24. Also shown in FIG. 13 is a programmable air pump 58. Although other pumps can be used, a peristaltic pump seems ideal. The air flow and the reagent can be independently programmed.

This pump supplies all positions on the multi tube rack. Each position on the multi tube ESR rack 24 will have its own pinch valve 56.

FIG. 14 shows the technician has “charged” the flexible pipette 14 and has inserted the sample tube 32. At the same time the pinch valve 56 remains closed and the pump 58 is off.

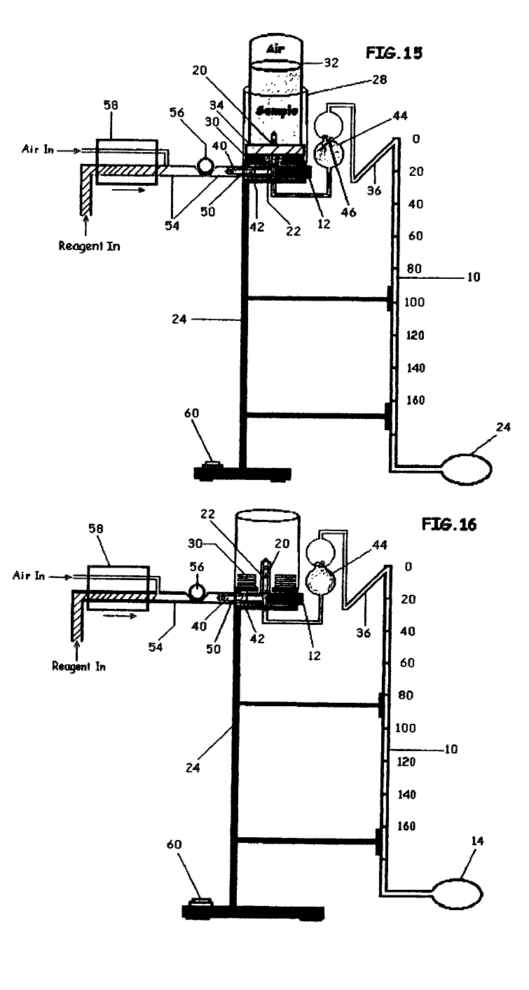

Immediately following the insertion of the sample tube 32 the sample will begin to flow. Several seconds later, as shown in FIG. 15, the sample will reach the mark 46 between the dual chambers 44. The technician then removes the sample tube 32. At that time the flow of the sample stops since the sample port 18 is sealed off. This is shown in FIG. 16.

As seen in FIG. 17 the technician now pushes a button switch 60 which activates the pinch valve 56 to open it and starts the pumping profile for reagent and air addition. This pumping profile will also take several seconds. The volume of the sample trapped in the lower dilution/mixing chamber is approximately 1000 microliter.

The reagent and air flow start pumping simultaneously. As the reagent and air flows into the reagent inlet tube 40 the flexible pipette 14 will insure this flow proceeds into the dilution/mixing chambers 44. Also, shown in FIG. 17 are air bubble segments 62 formed in the flowing stream. These bubble segments 62 will insure all blood sample is pushed into the dilution/mixing chambers 44. They will also act as mixing devices as they float up through the dilution/mixing chambers 44.

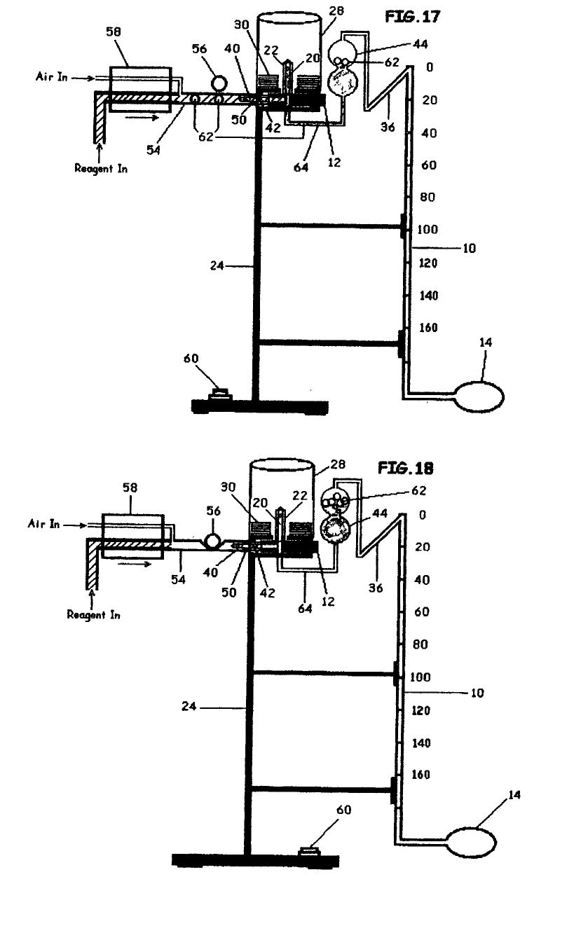

Near the end of the reagent/air pumping profile the programmed reagent pumping will stop and the air pumping will continue. As shown in FIG. 18, this will empty the conduit 64 between the pump 58 and the dilution/mixing chambers 44 and also create more air bubbles 62 to form and float up into the dilution/mixing chambers 44 for mixing. Then air pumping stops. This pumping profile takes several seconds. The volume of reagent pumped is approximately 250 microliters for a dilution of 4 parts sample and 1 part reagent.

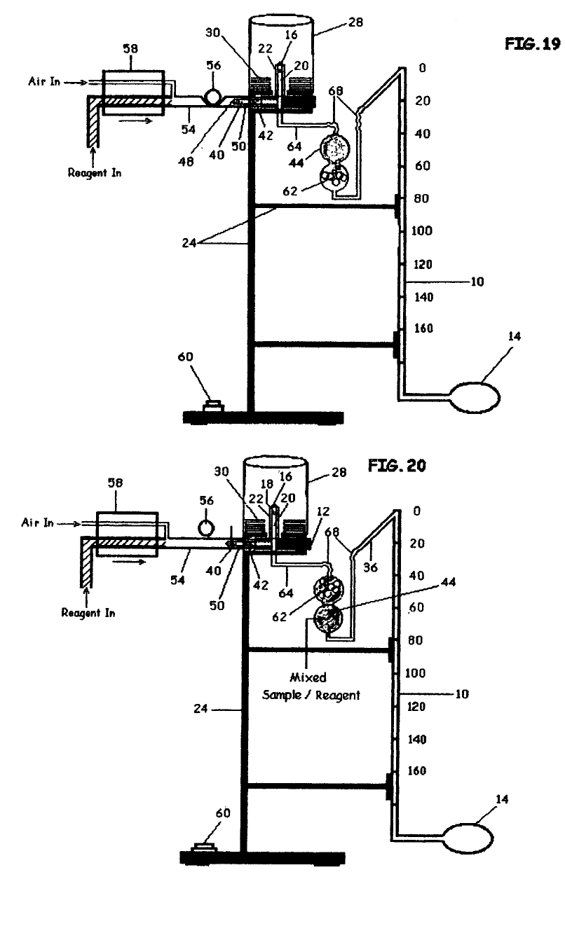

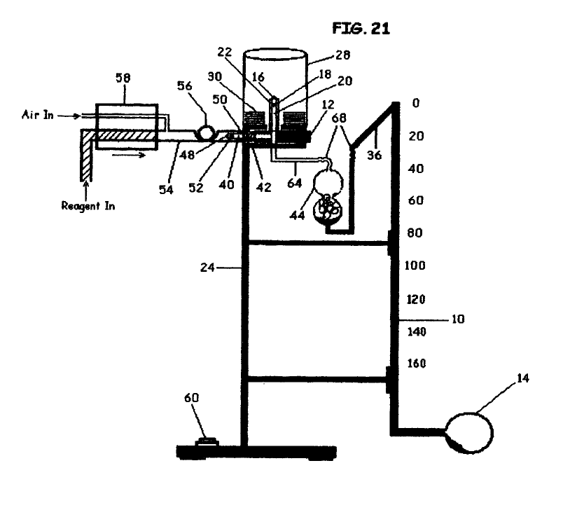

Referring to FIG. 19, upon completion of the pumping profile the technician will flip over the dilution/mixing chambers 44 180 degrees using their finger. The flexible pivot conduits 68 are shown in this drawing. This action will both create further mixing as the bubbles 62 rise back up through the dilution/mixing chambers 44 and create a direct path of the mixed sample/diluent solution to the ESR tube 10. FIG. 20 shows the air bubbles 62 have risen to the top of the “now” upper chamber 44. The technician will again push the button switch 60 which will both open the pinch valve 56 and start the air pump 58 which will cause air to flow through the reagent/air input tube 40 and allow the flexible pipette 14 to pull the mixed sample/reagent, filling the ESR tube 10. Then the pinch valve 56 will close again and the air pump 58 will stop as shown in FIG. 21. The ESR test will now begin. The technician will read the results one hour later, then discards the one piece ESR tube 10. It is obvious that the flipping over of the dilution/mixing chambers 44 can be automated mechanically. The invention drawing's starting with FIG. 22 and ending with FIG. 29 shows a method of performing other blood tests or chemical tests which will improve hazardous material handling in the laboratory.

FIG. 22 shows a one piece disposable test device 94, mostly rigid series of conduits, chambers 72 and 74, sampling pipette 76, and sampling tab 12. The reagent/air pipette 78 and the sample pipette 76 are flexible bulbs which act as suction pumps after being charged by the Technician squeezing them closed. The sampling tab 12 is exactly like the one shown in more detail in FIG. 10. Two other flexible conduits 80 and 81 are short sections before the two pipettes 76 and 78. The thickness of the conduit walls are shown to be very thin. Actually the wall thickness may be significantly thicker. It is designed to mix 10 microliters of sample with 200 microliters of reagent for a dilution of 1 to 20. “Point A” is the intersection where sample and reagent will combine. “Point B” is shown at the intersection below the chamber 72. The volume of the conduit 98 between these points is exactly 10 microliters. The volume of the lower chamber 72 is exactly 200 microliters. Also shown is a “mark” 82 between the two chambers 72 and 74.

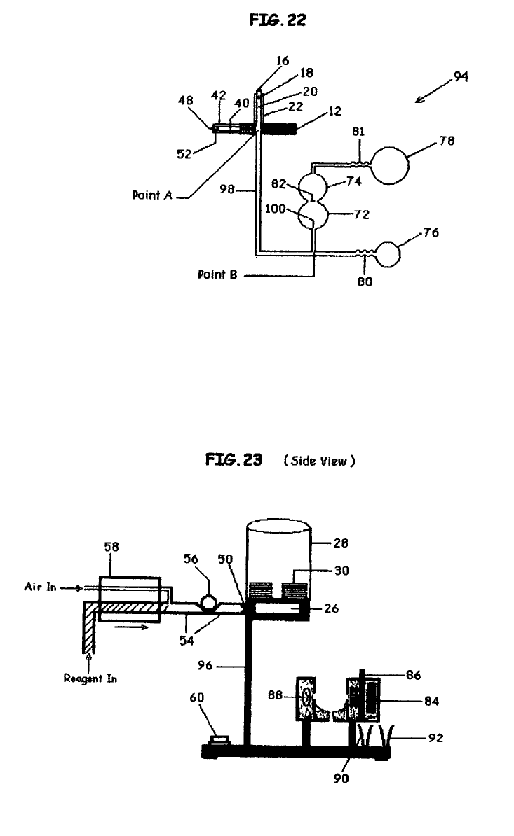

FIG. 23 shows a reagent/air pump 58, flexible tubing 54, pinch valve 56, sample tube holder 28, rack/base stand 96, calorimeter 84 with optical filter 86 and light source 88 and tubing pinchers 90 and 92. The pump conduits for air and reagent flexible tubing 54 and pinch valve 56 are shown near actual size. The tubing pinchers 90 and 92 are near actual size and are mounted to the rack/base stand 96.

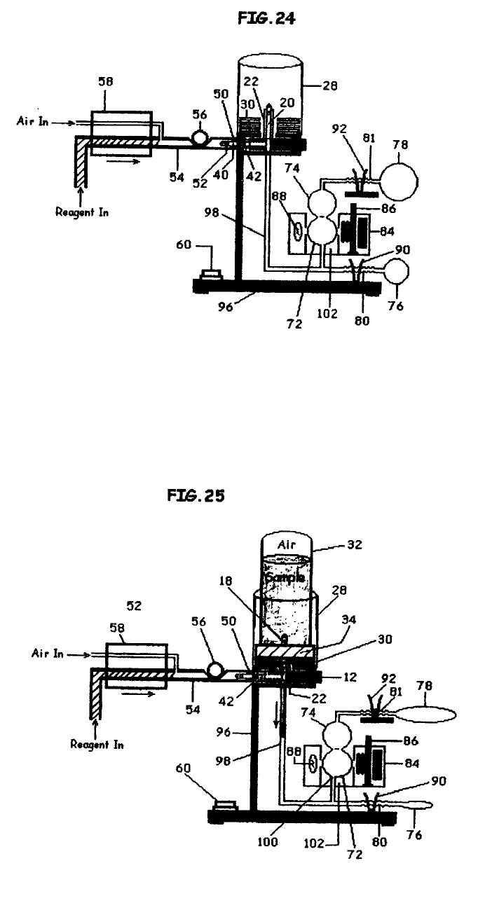

FIG. 24 shows the Technician has placed the test device 94 shown in FIG. 22 into position for testing. The sampling tab 12 and sample tube holder 28 are the same as shown in more detail in FIGS. 9-12. The upper, or reagent/air tubing pincher 92 is actually mounted on the rack/base stand 96 as shown in FIG. 23. It is also drawn up higher in FIGS. 24-28 only to reduce the drawing complexity.

In the first step before placing the disposable device 94 in position, the Technician will squeeze the reagent/air pipette 78 and sample pipette 76 to “charge” them. Then the technician will push the disposable device 94 into position and will push the flexible tubing 80 and 81 down into the tubing pinchers 90 and 92.

As the pipettes 76 and 78 are “charged” they will create air pressure which will escape by pushing open the silicone sleeves 22 and 42 which surround the sample inlet port 18 and the reagent/air inlet port 52. As shown in FIG. 25 the Technician then pushes a sample tube 32 down into the sample tube holder 28, releases the flexible tubing 80 in front of the sample pipette 76 from the tubing pincher 90.

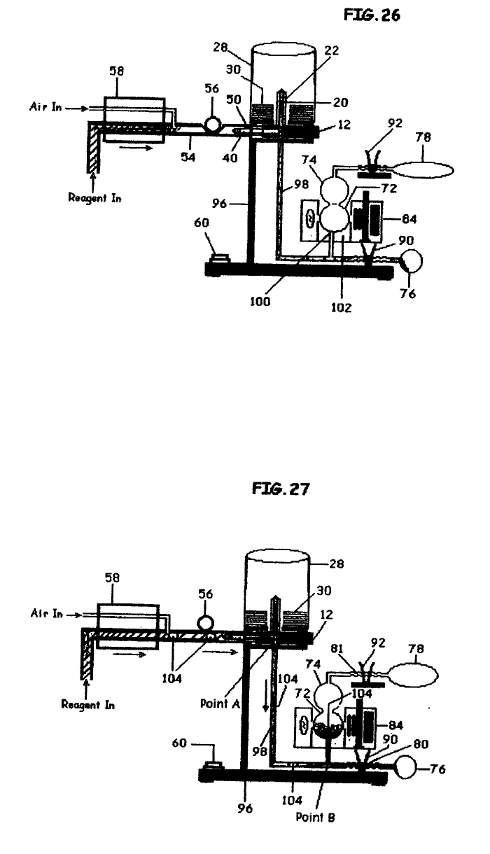

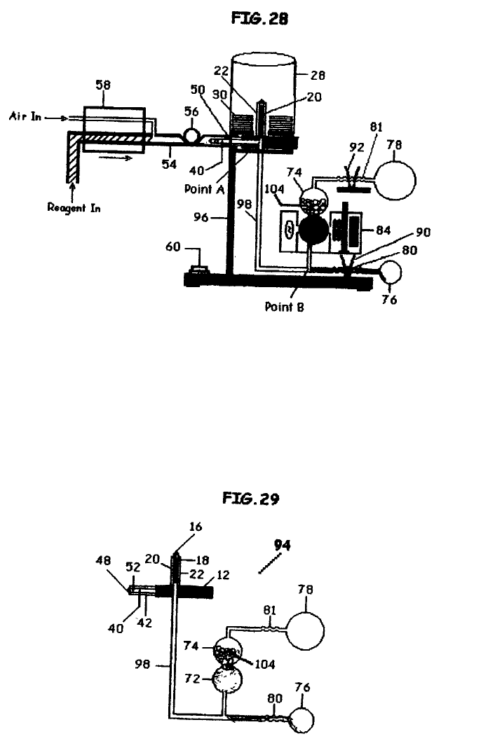

Referring to FIG. 26, when the sample flow has reached the sample pipette 76 the technician will again push the flexible tubing 80 down into the sample pincher 90, sealing off all conduits, chambers and pipettes. The Technician then removes the sample tube 32. Referring to FIG. 27, the Technician now removes the flexible tubing 81 from the reagent/air pincher 92 and pushes the pump profile switch 60. The pinch valve 56 opens and the pumping profile begins. Air bubbles 104 are injected into the flowing reagent stream. This flowing stream displaces the trapped sample between Points A and B as shown in FIG. 22. This flowing stream proceeds to fill the lower mixing, dilution and measurement chamber 72. As the bubbles 104 float up through this chamber 72 they mix the sample and reagent. The reagent profile pump 58 will pump approximately 200 microliters and then stop. The air profile pump 58 will continue pumping and displacing all reagent within the conduit 98 path into the lower chamber 72 to fully mix the sample and reagent. The conduit entry port 100 leading into the bottom chamber 72 may need to be off-set to one side to insure complete mixing with certain tests. Also, the upper chamber 74 may need to be larger to accept more air bubbles.

FIG. 28 shows a completed test. Although some tests can be measured by the colorimeter immediately, other tests will need more reaction time or heat to be complete. Delay time will be provided before measurement. The colorimeter 84 base 102 can be a heater for those tests needing heat to complete a reaction.

FIG. 29 illustrates the one piece disposable test device 94 with sample, air and reagent entrapped.

FIG. 30 shows a modified test device 106 having an additional tab 112. This tab 112 is identical to the sampling tab 12 shown in the prior drawings FIGS. 1 and 2. It has a inlet tube 120 with a silicone sleeve 122 and inlet port 118.

This tab 112 is for an alternate vacuum source to pull sample, air and reagent through the conduits.

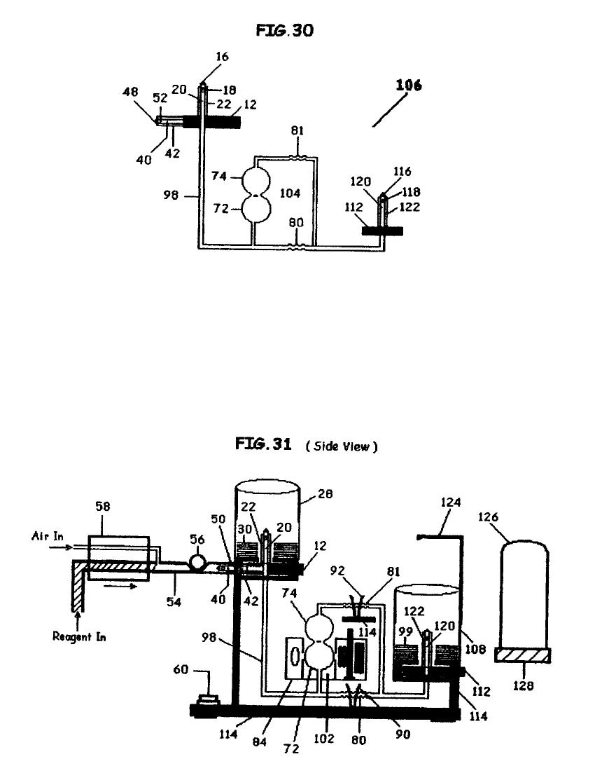

FIG. 31 shows the alternate vacuum source 126 which is a common vacuum sampling tube 126 with a rubber stopper 128. This vacuum tube 126 replaces the sampling pipette 76 and reagent/air pipette 78 shown in FIGS. 22 through 29. FIG. 31 also shows a second tube holder 108 mounted to the rack base stand 114. This tube holder 108 is identical to the sample tube holder 28 shown in FIG. 3,4 and 5, except it has a tube keeper 124 that keeps the vacuum tube 126 in place during operation. This tube keeper 124 will adjust vertically to accept various length vacuum tubes 126 and pivot horizontally to permit the Technician to install and remove the vacuum tube 126. This vacuum tube 126 offers higher vacuum pressure and more vacuum capacitance. Also, multiple tests may be performed with a single vacuum tube 126. The Technician places the test device 106 in position on the instrument then pushes the flexible conduits 80 and 81 down into the tubing pinchers 90 and 92. The Technician then pushes the vacuum tube 126 down into the vacuum tube holder 108 and moves the tube keeper 124 in place to hold the vacuum tube 126 in place. The Technician then continues the instrument operation as discribed earlier. Following completion of the test the Technician will remove the vacuum tube 126. The tubing pinchers 90 and 92 can be replaced with programed automatic pinch valves to further automate the tests. This alternate vacuum source can also be used for the ESR test described earlier.

While the preferred embodiments have been herein disclosed, it is understood and acknowledged that variation can be made without departing from the scope of the invention as claimed.

What is claimed is:

1. A system for handling fluids and gases, said system comprising
a generally rigid, generally elongated tube having an internal passage of a generally constant diameter;
a selectively openable and closeable first port at a first end of said tube in fluid communication with said internal passage; and
a pressure source in communication with said internal passage adapted to provide positive or negative pressure to said internal passage thereby causing a liquid or gas to be drawn into or expelled from said internal passage via said first port.

2. A system according to claim 1, wherein
said pressure source is a flexible bulb.

3. A system according to claim 1, wherein
said first port comprises a generally tube-shaped body having a hole therethrough and a flexible sleeve covering said hole to seal shut said first port, said sleeve being adapted to slide away from said hole to open said first port.

4. A system according to claim 3, wherein
said first port is adapted to penetrate a seal on a container of liquid or gas causing said container to slide said sleeve away from said hole to enable said liqiuid or said gas to be drawn into said internal passage from said container.

5. A system according to claim 1, further comprising
a plurality of generally evenly spaced apart, visual indicia markings positioned along the outer surface of said tube.

6. A system according to claim 1, further comprising
a support structure adapted to hold said tube stationary relative to and upon a work surface.

7. A system according to claim 6, further comprising
a sample container holder attached to said support structure and adapted to hold a sample container containing a gas or liquid in a position such that said first port is in fluid communication with the contents of said sample container when said tube is held by said support structure.

8. A system according to claim 7, further comprising
at least one spring assembled within said sample container holder and adapted to be energized when said sample container is placed in said sample container holder and further adapted to release energy when said sample container is removed from said sample container holder in a manner to provide force to assist such removal of said sample container.

9. A system according to claim 1, further comprising
at least one mixing chamber having a diameter greater than the diameter of said tube, said mixing chamber being in fluid communication with and being positioned in series between said first port and said pressure source.

10. A system according to claim 1, further comprising
a second port in communication with said internal passage and having a fluid delivery source for delivering gas or liquid, under pressure, to said internal passage.

11. A system according to claim 10, further comprising
a motor for powering said fluid delivery source.

12. A system according to claim 10, further comprising
a valve positioned between said second port and said fluid delivery source and being adapted to control flow of gas or liquid into said internal passage via said second port.

13. A system according to claim 10, further comprising
a second pressure source in communication with said internal passage adapted to provide positive or negative pressure to said internal passage thereby causing a liquid or gas to be drawn into or expelled from said internal passage via said first port or said second port.

14. A method of testing a fluid substance, said method comprising
providing a generally rigid, generally elongated tube having an internal passage of a generally constant diameter and being generally transparent;
providing a selectively openable and closeable first port at a first end of said tube in fluid communication with said internal passage;
providing a pressure source in communication with said internal passage adapted to provide positive or negative pressure to said internal passage;
activating said pressure source thereby causing a liquid or gas to be drawn into said internal passage via said first port; and
visually inspecting the liquid or gas through the external surface of said tube.

15. A method according to claim 14, wherein
a plurality of generally evenly spaced apart, visual indicia markings are positioned along the outer surface of said tube.

16. A method according to claim 14, further comprising
positioning a sample container having a liquid or gas contained therein into fluid communication with said first port.

17. A method according to claim 16, further comprising
positioning a sample container and said tube into a support frame which holds said container and said tube stationary relative to each other while maintaining said first port in fluid communication with said container.

18. A method according to claim 14, further comprising
providing a second pressure source in fluid communication with said internal passage; and
activating said second pressure source thereby causing a liquid or gas to be drawn into said internal passage via said second port.

19. A method according to claim 14, further comprising
providing at least one mixing chamber having a diameter greater than the diameter of said tube, said mixing chamber being in fluid communication with and being positioned in series between said first port and said pressure source; and
activating said pressure source to cause said liquid or gas to be transported to said mixing chamber and mixed therein.

20. A method according to claim 17, further comprising
removing said sample container from said support frame;
positioning a different sample container on said support frame in fluid communication with said first port.

21. A method according to claim 16, further comprising
removing said tube from said support frame;
discarding said tube;
providing a second generally rigid, generally elongated tube on said frame, said second tube having an internal passage of a generally constant diameter and being generally transparent;
providing a second selectively openable and closeable port at a first end of said second tube in fluid communication with said second tube internal passage;
providing a second pressure source in communication with said second internal passage adapted to provide positive or negative pressure to said second internal passage;
activating said pressure source thereby causing a liquid or gas to be drawn into said second internal passage via said second port; and
visually inspecting the liquid or gas through the external surface of said second tube.

22. A method of testing a fluid to measure the content of a relatively dense substance in said fluid, said method comprising
providing a generally rigid, generally elongated tube having an internal passage of a generally constant diameter and being generally transparent;
providing a selectively openable and closeable first port at a first end of said tube in fluid communication with said internal passage;
providing a pressure source in communication with said internal passage adapted to provide positive or negative pressure to said internal passage;
activating said pressure source thereby causing said fluid to be drawn into said internal passage via said first port; and
visually inspecting said fluid through the external surface of said tube to determine the height within said tube of the highest vertical position of said relatively dense substance in relation to the level of said fluid.

23. A method according to claim 22, wherein
said fluid and said relatively dense substance comprise, respectively, plasma and red blood cells.
